(19) United States
(12) Patent Application Publication
(10) Pub. No.: US 2026/0130483 A1
(43) Pub. Date: May 14, 2026

(54) PHYSIOTHERAPY COMB DEVICE

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/277,488

(22) Filed: Jul. 23, 2025

(30) Foreign Application Priority Data
Nov. 12, 2024 (CN) 202422759131X

(51) Int. Cl. A45D 24/22; A45D 24/14; A61N 5/06; A61N 5/067

(52) U.S. Cl. CPC A45D 24/22; A45D 24/14; A61N 5/0617; A61N 5/067; A45D 2200/205; A61N 2005/063; A61N 2005/0644; A61N 2005/0651

(57) ABSTRACT

The present invention discloses a physiotherapy comb device designed to enhance user convenience and therapeutic efficiency. The device comprises a main body equipped with a liquid cavity, a liquid outlet, and a first vent hole, along with a sealing cover that features a second vent hole. The sealing cover is designed to slide between two positions to control air exchange, enabling easy sealing and unsealing of the device. The main body includes a movable groove that accommodates an elastic sealing portion of the cover, which is securely held in place using interlocking latching structures. A rolling element is rollably mounted at the liquid outlet to facilitate smooth topical application. The apparatus further includes multiple massage protrusions or comb-like teeth and may integrate an optional stimulation element such as phototherapy, hot or cold compress, microcurrent, or vibration for enhanced therapeutic outcomes.

### TECHNICAL FIELD

The present invention pertains to the field of therapeutic and personal care devices, and more particularly, to a physiotherapy comb device that integrates liquid dispensing functionality with improved pressure control to enhance the delivery and application of care solutions such as serums, essential oils, or treatment liquids.

### BACKGROUND

Physical therapy devices are increasingly used in home and clinical environments to promote health, manage chronic conditions, and enhance personal wellness routines. These devices often utilize care solutions, such as essential oils, medicinal serums, or scalp treatments, to improve therapeutic outcomes. For example, hair care combs may be used with growth serums to stimulate follicles, while massage devices are commonly paired with oils to moisturize the skin and aid absorption during treatment sessions.

To preserve the effectiveness of such care solutions, it is critical to store them in airtight containers to prevent oxidation, evaporation, or contamination. However, ensuring both airtight storage and smooth dispensing remains a technical challenge. Conventional designs often use sealed containers with small liquid outlets, which prevent air from entering during use. This creates an internal vacuum effect that hinders fluid flow, resulting in a poor user experience.

Existing devices typically rely on squeezing, tilting, or shaking the container to dispense the care liquid, which is inconvenient and often ineffective. Some advanced models incorporate pump systems or complex valve mechanisms, but these increase the cost, complexity, and likelihood of mechanical failure. Furthermore, they may still fail to maintain a fully sealed environment when not in use, compromising the long-term stability of the care solution.

Current solutions do not adequately balance the need for airtight storage with the practical requirement of easy, gravity-assisted liquid flow. There is a lack of simple, user-friendly mechanisms that can reliably allow air into the container only when needed, without compromising the seal when the device is idle.

In order to overcome these limitations, the present invention provides a physiotherapy comb device with an integrated air control mechanism that enables controlled venting through a movable sealing cover. The design allows external air to enter the sealed liquid cavity only when the user slides the cover into an open position, equalizing pressure and enabling smooth dispensing of the care solution. When not in use, the cover returns to a closed position, maintaining airtight conditions. This design significantly improves the ease of use, effectiveness, and preservation of care solutions in physiotherapy comb devices.

### OBJECTS OF THE INVENTION

Some of the objects of the invention are as follows:

An object of the present invention is to provide a multifunctional physiotherapy comb device capable of dispensing care solutions such as hair serums, scalp tonics, or other therapeutic liquids in a controlled manner, using an integrated vent-controlled liquid chamber.

Another object of the present invention is to provide a physiotherapy comb device having a sliding sealing cover with a selectively alignable vent hole, which allows controlled entry of air into the liquid chamber for smooth and gravity-assisted dispensing of the care solution.

Another object of the present invention is to provide a physiotherapy comb device incorporating a rolling element at the liquid outlet, configured to rotate and evenly spread the dispensed solution across a user’s skin or scalp, enhancing absorption and comfort.

Another object of the present invention is to offer a comb structure with tactile feedback features such as latching or detent mechanisms along the sealing cover’s sliding path, allowing users to intuitively sense and switch between open, closed, and intermediate flow states.

Another object of the present invention is to provide a physiotherapy comb device with dual liquid containers and a multi-position sealing mechanism that enables users to selectively dispense from either of the containers using a single actuation path.

Another object of the present invention is to enhance functionality by incorporating one or more stimulation elements, including but not limited to a phototherapy lamp (Light Emitting Diodes (LEDs), or lasers elements), heating elements, magnetic element, cooling elements, ultrasonic wave element, vibratory elements, electrodes, etc., to support advanced therapeutic applications such as phototherapy, electrotherapy, micro-current therapy, piezoelectric therapy, ultrasonic wave therapy, thermal therapy, or vibration therapy.

Another object of the present invention is to provide comb teeth with integrated microchannels for directing liquid flow to the treatment area, optionally fabricated from light-transmitting polymers to allow targeted delivery of light energy from internal stimulation elements.

Another object of the present invention is to provide a hygienic and modular physiotherapy comb device wherein the comb teeth are detachably connected to the main body using magnetic or snap-fit couplings, enabling easy cleaning and customization.

Another object of the present invention is to offer a compact, ergonomic, and user-friendly physiotherapy comb device suitable for both dry stimulation and wet care applications, improving the efficiency and convenience of personal grooming and scalp therapy routines.

### SUMMARY OF THE INVENTION

According to a first aspect of the present invention, a multifunctional physiotherapy comb device is provided. The device comprises: a main body including a housing and a liquid chamber configured to store a care solution, the liquid chamber being in fluid communication with a liquid outlet and a first vent hole; a sealing cover slidably mounted on the housing, the sealing cover comprising a sealing part and a trigger part and further including a second vent hole; wherein the sealing cover is movable between a first position and a second position. In the first position, the second vent hole aligns with the first vent hole to permit airflow into the liquid chamber, enabling gravity-assisted dispensing of the care solution through the liquid outlet. In the second position, the second vent hole is offset from the first vent hole, thereby sealing the chamber and restricting liquid discharge. A rolling element is rotatably mounted on the housing near the outlet and is configured to spread the care solution on the scalp or skin surface as it rotates. The device further includes at least one stimulation element embedded within the housing to deliver therapeutic treatment during use.

In one embodiment of the invention, the main body comprises an active groove formed on its outer surface, and the sealing part of the sealing cover is configured to be received within and slide along the active groove in a first direction. The active groove includes opposing first and second groove side walls that elastically clamp the sealing part, thereby restricting displacement in a direction perpendicular to the sliding path. The groove further includes third and fourth side walls disposed along the direction of motion, wherein the sealing part abuts the third side wall in the first position and the fourth side wall in the second position.

In one embodiment, the first groove side wall includes a first latching protrusion, while the sealing part includes a second latching protrusion, wherein the two protrusions cooperate to provide tactile resistance and positional stability during sliding movement. The sealing cover may be formed by dual-material injection molding, with the sealing part made from an elastomeric material and the trigger part formed of a rigid structure. A ring-shaped protrusion of the trigger part is embedded in a ring groove of the sealing part formed between a center and edge protrusion, providing a secure structural interlock.

In one embodiment, the rolling element is a spherical massage ball or a cylindrical element, rotatably mounted within a recessed socket of the main body. The rolling element is positioned adjacent to the liquid outlet and configured to dispense and evenly distribute the care solution across the user’s scalp or skin surface during movement of the comb. The first vent hole may have a larger diameter than the second vent hole and may also serve as a refill port when the sealing cover is removed.

According to a second aspect of the invention, a physiotherapy comb device is provided comprising a main body that houses a first liquid container and a second liquid container, each configured to store different care solutions. The first container is in communication with a first liquid outlet and a first vent hole, while the second container is in communication with a second outlet and a second vent hole. A common sealing cover is slidably mounted on the housing and movable between a first position, a second sealed position, and a third position. In the first position, the sealing cover aligns a third vent hole with the first vent hole to allow dispensing of the first care solution. In the third position, the third vent hole aligns with the second vent hole to enable dispensing from the second container. In the sealed second position, the third vent hole is offset from both the first and second vent holes, thus sealing both chambers. A rolling element is positioned near the respective liquid outlets to assist in dispensing the selected solution during application. The rolling element can be a circular ball that rolls to provide a massage effect.

In one embodiment, the first and second liquid containers are removably mounted within the housing and are individually refillable or replaceable. Each outlet may be paired with a corresponding rolling element to enable selective application of each care solution. The sealing cover is configured to slide within an active groove formed on the housing and may include a detent or latching mechanism to provide tactile feedback upon transitioning between positions. In one embodiment, the sealing part is circular and slides within an elliptical groove having a long axis aligned with the sliding direction, thereby ensuring linear motion while preventing lateral displacement.

According to a third aspect of the invention, the physiotherapy comb device further comprises a plurality of comb teeth extending from the housing. The comb teeth may include grooves or microchannels along their length to guide the dispensed liquid toward the user's scalp or skin surface. The teeth may be formed of a light-transmitting polymer to enable delivery of phototherapy directly to the treatment site. In one embodiment, the comb teeth are detachably mounted to the main body using a magnetic or snap-fit coupling, enabling ease of cleaning or replacement with teeth of various types.

According to a fourth aspect of the invention, a method of performing a therapeutic treatment using the physiotherapy comb device is provided. The method comprises: positioning the sealing cover in the first position to align the vent holes and allow airflow into the liquid chamber, thereby facilitating liquid flow through the outlet; rotating the rolling element against the scalp or skin to dispense and spread the care solution; and optionally activating one or more embedded stimulation elements. These elements may include a light-emitting diode (LED), micro-current electrode, magnetic element, vibration element, and heating or cooling element to provide enhanced therapeutic effects. In another embodiment, the method further comprises guiding the dispensed solution along grooves or microchannels formed in the comb teeth, with light from the stimulation element traveling through light- transmitting comb teeth to perform localized phototherapy during application.

In one embodiment of the invention, the comb teeth are fabricated from a transparent or translucent polymer that enables transmission of light from the stimulation element to the user’s skin.

In one embodiment of the invention, the comb teeth are formed with anti-bacterial or silicone-coated surfaces to enhance hygiene and skin compatibility during repeated use.

In one embodiment of the invention, the comb teeth are detachably mounted to the main body via a snap-fit or magnetic coupling structure, enabling easy cleaning or replacement with comb teeth of varying shapes or sizes.

In one embodiment of the invention, the at least one stimulation element includes but is not limited to a phototherapy lamp, a hot compress element, a cold compress element, a microcurrent element, or a vibration element.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term "several" means more than one, unless otherwise specified.

In the context of the specification, the term "LED" refers to one or more light-emitting diode (LED) elements that are electrically connected and configured to emit light of specific wavelengths (including visible to IR wavelengths) suitable for therapeutic purposes. The LED element or module may include drive circuitry, heat dissipation structures, and optical elements such as lenses or diffusers to control light distribution.

In the context of this specification, terms like “light,” “radiation,” “irradiation,” “emission” and “illumination,” etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorised into several categories according to respective wavelength ranges, which are again envisaged to be within the scope of this invention. A commonly used subdivision scheme for IR radiation includes Near IR (0.75-1.4μm), Short-Wavelength IR (1.4-3μm), Mid-Wavelength IR (3-8μm), Long-Wavelength IR (8-15μm), and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for the treatment of conditions that occur at the skin surface, such as psoriasis or infection.

In the context of the specification, the term “light source” or “phototherapy source,” etc., refers to a source emitting coherent laser light, or light-emitting diodes (“LEDs”). The term “light therapy” refers to light generated from any of the sources, such as lasers, LED sources, or Super luminous diodes (“SLD”), or Organic light-emitting diodes (OLED).

In the context of the specification, the term "lamp board" refers to a printed circuit board or similar substrate on which one or more light sources are mounted and electrically connected to a power source, such as a rechargeable battery.

In the context of the specification, the term "light source" refers to any active component, such as a light-emitting diode (LED), capable of emitting phototherapy light (e.g., red, infrared, or blue) toward the scalp.

In the context of the specification, the term "light transmission hole" refers to an aperture formed in the housing of the second rotating arm, aligned with the light source to allow therapeutic light to exit the comb and reach the scalp.

In the context of the specification, the term "light transmission plate" refers to a transparent or translucent cover positioned over the light transmission hole to protect internal components while allowing light to pass through.

In the context of the specification, the term "pressing switch" refers to a mechanical actuator disposed on the surface of the comb and configured to trigger an internal switching device that activates the lamp board or associated electronics.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colours), Surface Mount Technology (SMT) LEDs, Bi-colour LEDs, Pulse Width Modulated RGB (Red-Green-Blue) LEDs, and high-power LEDs, among others. The LEDs may also include UV-emitting diodes specifically selected for antimicrobial action in disinfection modules integrated into the multifunctional physiotherapy comb device.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminium, Boron, Zinc Selenide, etc., in pure form or doped with elements such as Aluminium and Indium etc. For example, red and amber colours are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider colour gamut. White and other colored lightings may also be produced using phosphor coatings such as Yttrium Aluminium Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the target surfaces. TENS therapy uses low-voltage currents to provide pain relief. Electrical impulses are delivered through electrodes placed on the surface of the body of the user. The electrodes are placed at or near the comb teeth.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a top perspective view of a physiotherapy comb device, in accordance with an embodiment of the present invention.

FIG. 2 illustrates a bottom perspective view of a physiotherapy comb device, in accordance with an embodiment of the present invention.

FIG. 3 illustrates a top view of a physiotherapy comb device, in accordance with an embodiment of the present invention.

FIG. 4 shows a cross-sectional view of the physiotherapy comb device along plane A-A’ of FIG. 3, in accordance with an embodiment of the present invention.

FIG. 5 shows an enlarged view of a given point B of the physiotherapy comb device illustrated in FIG. 4, in accordance with an embodiment of the present invention.

FIG. 6 illustrates a cross-sectional view of the physiotherapy comb device depicting multiple liquid chambers and positions of the sealing cover, in accordance with an embodiment of the present invention.

FIG. 7 illustrates an exploded view of a main body and a sealing cover of the physiotherapy comb device, in accordance with an embodiment of the present invention

FIG. 8 illustrates a top view of a first vent hole of the physiotherapy comb device, in accordance with an embodiment of the present invention.

FIG. 9 illustrates an exploded view of the physiotherapy comb device, in accordance with an embodiment of the present invention.

FIG. 10 illustrates an exploded top view of the sealing cover and a sealing part of the physiotherapy comb device, in accordance with an embodiment of the present invention.

FIG. 11 illustrates an exploded bottom view of the sealing cover and a sealing part of the physiotherapy comb device, in accordance with an embodiment of the present invention.

FIG. 12 illustrates an exploded cross-sectional view of the sealing cover and a sealing part of the physiotherapy comb device, in accordance with an embodiment of the present invention.

FIG. 13 is a flowchart showing a method of using the physiotherapy comb device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

Embodiments of the present invention relate to a physiotherapy comb device and a method for performing therapeutic treatment using such a device. More particularly, the invention concerns a hand-held comb-like applicator that dispenses a care solution onto a user’s skin or scalp while simultaneously delivering stimulation therapy such as phototherapy, vibration, microcurrent stimulation, or thermal treatment. The device is particularly suited for applications in dermatology, scalp therapy, pet care, or wellness treatment..

In an embodiment, the physiotherapy comb device comprises a main body including a housing and at least one liquid chamber configured to store a care solution. The liquid chamber is in communication with a liquid outlet and a first vent hole. A sealing cover is slidably mounted on the main body, the sealing cover comprising a sealing part and a trigger part, and includes a second vent hole. The sealing cover is configured to slide along a predefined direction, typically a linear guide path formed on the main body, between a first position and a second position. In the first position, the second vent hole aligns with the first vent hole, thereby allowing ambient air to enter the liquid chamber and facilitating gravity-assisted dispensing of the care solution through the liquid outlet. In the second position, the second vent hole is offset from the first vent hole, effectively sealing the liquid chamber and preventing further outflow of the care solution. The slidable vent control allows the user to initiate or stop dispensing with minimal effort and without relying on complex valve mechanisms.

The outer surface of the main body may include an active groove, within which the sealing part of the sealing cover is received and constrained for sliding motion. Opposing side walls of the groove elastically clamp the sealing part in a direction perpendicular to the sliding direction, ensuring structural stability and resistance to unintentional displacement. In certain embodiments, the groove may include clamping or latching protrusions that cooperate with mating structures on the sealing part, thereby providing tactile resistance or a detent-like feedback as the sealing cover transitions between positions. This enhances user feedback and confirms actuation without requiring complex locking mechanisms.

A rolling element, preferably spherical or cylindrical in shape, is rotatably mounted in the housing adjacent to the liquid outlet. The rolling element rotates in response to motion across the treatment surface and is positioned to receive the dispensed care solution from the outlet. As it rotates, the rolling element spreads the solution evenly while also providing mechanical massage. The rolling element may be partially exposed through a first opening in the housing and seated in a socket that maintains fluid contact with the outlet.

To enhance therapeutic efficacy, the device further comprises at least one stimulation element embedded within the housing. The stimulation element may include, for example, a phototherapy module comprising one or more light-emitting diodes (LEDs) or a laser, a piezoelectric element, a microcurrent element with low-voltage electrodes, a vibration motor, an ultrasonic wave therapy element, or a thermal treatment module capable of heating or cooling the skin-contact region. These stimulation elements can work individually or in combination to target specific treatment goals like promoting blood circulation, relieving inflammation, or enhancing product absorption. The comb teeth may be formed with micro-channels to direct fluid flow and can be fabricated from light-transmitting materials to enable localized delivery of phototherapy. The teeth are optionally detachable for hygienic cleaning or customization. With its ergonomic design, intelligent control options, and versatile therapeutic capabilities, this comb device offers a comprehensive solution for both home and clinical scalp or skin treatment routines.

The main body may further include a plurality of comb teeth extending from its surface. The comb teeth may be integrally formed or detachably mounted, and may be configured with longitudinal microchannels or grooves that facilitate directional flow of the care solution from the outlet toward the user's scalp or skin. In certain configurations, the comb teeth are made of transparent or light-transmitting materials to allow light emitted from the embedded LEDs to reach the treatment area directly or via internal reflection. This structure enables simultaneous phototherapy and topical application of the care solution, enhancing the therapeutic outcomes. In some embodiments, the comb teeth are fabricated from antimicrobial polymers or coated with silicone to enhance skin compatibility and hygiene.

In an embodiment, the first vent hole may also serve as a refill port. The sealing cover is configured to be detachable from the main body, thereby exposing the first vent hole and allowing the user to replenish the liquid chamber with care solution directly. This configuration eliminates the need for a separate filling mechanism, simplifying the overall design. The sealing part may be formed from an elastomeric material such as silicone or rubber to provide effective sealing, while the trigger part may be constructed from a rigid plastic such as ABS or polycarbonate. These components may be joined using mechanical assembly or fabricated together using dual-material injection molding technology to combine flexibility and rigidity in a single integrated structure.

In another embodiment of the invention, the physiotherapy comb device includes a dual liquid chamber configuration, enabling selective dispensing of two different care solutions. The main body comprises a housing that accommodates a first liquid container and a second liquid container, each defining a respective liquid chamber. The first container includes a first liquid outlet and a first vent hole, while the second container includes a second liquid outlet and a second vent hole. A single sealing cover is slidably mounted on the housing and is movable between at least three discrete positions. In the first position, a third vent hole formed in the sealing cover aligns with the first vent hole, allowing air inflow into the first liquid container and enabling gravity-fed dispensing of the first care solution through its corresponding outlet. In the second position, the third vent hole is offset from both vent holes, sealing both containers and preventing liquid discharge. In the third position, the third vent hole aligns with the second vent hole, allowing airflow into the second liquid container and enabling dispensing of the second care solution through the second outlet.

The rolling element in this embodiment may be positioned adjacent to the outlets of both containers and is configured to receive and distribute whichever solution is selected via the sealing cover’s position. Alternatively, two separate rolling elements may be provided, each aligned with its respective outlet, to maintain the separation of solutions and enable clean switching between treatments. This embodiment is particularly advantageous for alternating between therapeutic agents, such as applying a hydrating solution followed by an anti-inflammatory serum, or alternating between human-use and pet-care formulations.

In some embodiments, the first and second liquid containers are removably mounted within the housing and configured to be refillable or replaceable. This modular design enables users to customize treatment based on need and facilitates cleaning or replacement. The sealing cover may still be received in an active groove, with additional detents provided to define three stable positions for the dual-chamber operation. Each container may also include a uniquely dimensioned outlet or keying feature to ensure proper orientation and fluid control.

In both single and dual container embodiments, the sealing cover may be constructed via dual-material injection molding, with the sealing part formed of a soft elastomer and the trigger part made of rigid thermoplastic. The sealing part may include a center bulge and edge bulge forming a groove to receive a ring protrusion from the trigger part. This configuration ensures mechanical integrity and sealing performance during repeated sliding.

The invention also provides a method for performing therapeutic treatment using the physiotherapy comb device. The method includes the steps of providing a device comprising a liquid chamber, a sealing cover with a second vent hole, and a rotatable rolling element; sliding the sealing cover to a first position in which the second vent hole aligns with the first vent hole to allow air inflow into the chamber; and applying the rolling element to the treatment surface to dispense the care solution through the outlet. As the device is moved across the skin or scalp, the rolling element rotates, thereby spreading the solution uniformly. The method may further include activating one or more stimulation elements during dispensing, including LED light emission, microcurrent stimulation, vibratory massage, or thermal modulation. These stimulation features can be operated individually or in combination, based on user preference or therapeutic protocol.

In an embodiment, the device may incorporate a control module with wireless communication capabilities, such as Bluetooth or Wi-Fi, allowing it to connect to an external user interface or mobile application. Through this interface, users may adjust treatment intensity, monitor usage statistics, activate specific stimulation features, and configure custom treatment programs. This transforms the physiotherapy comb into a smart therapeutic tool suitable for both consumer wellness and clinical applications.

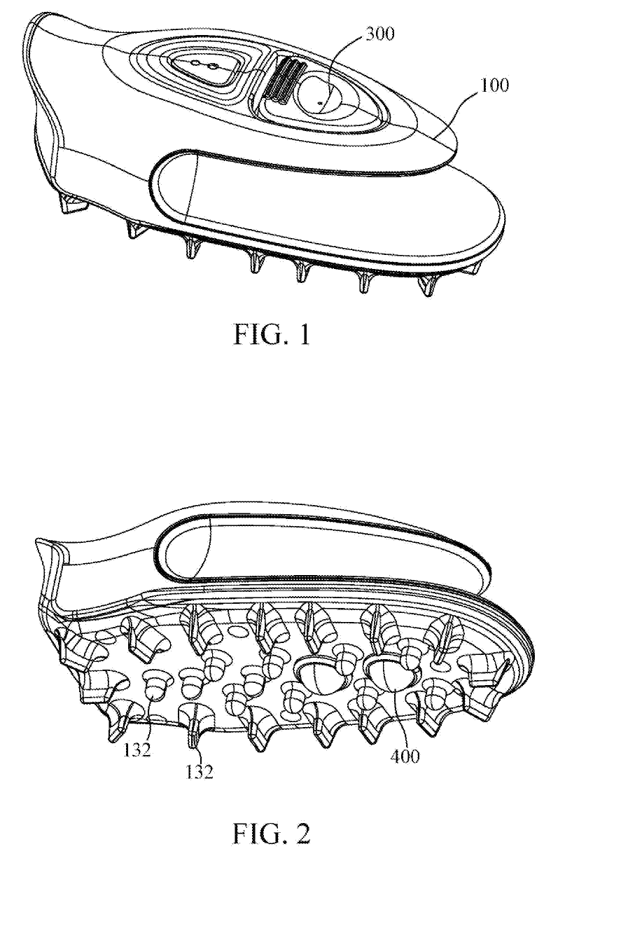

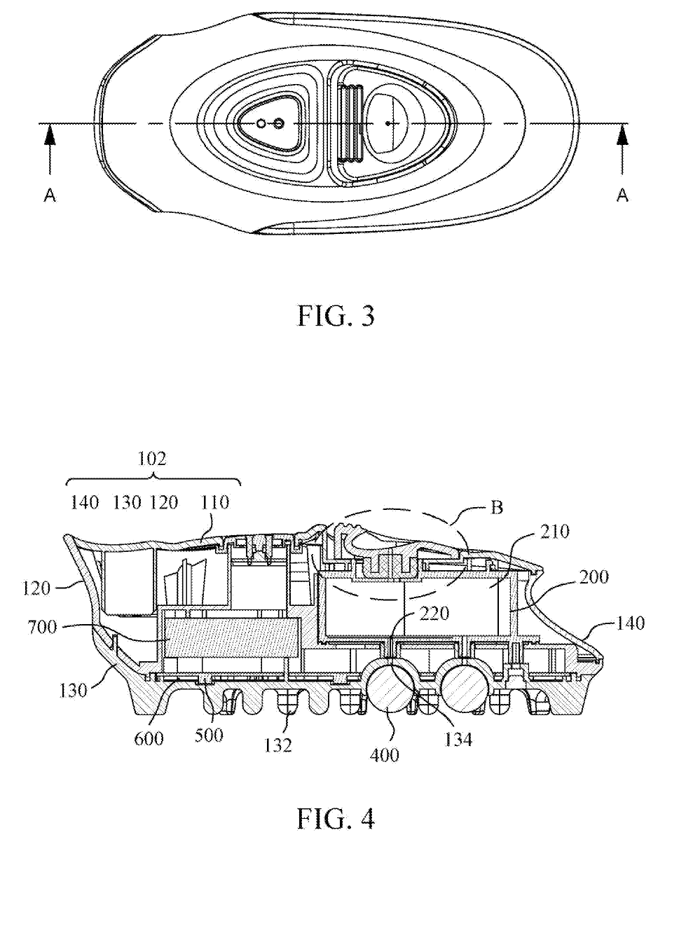

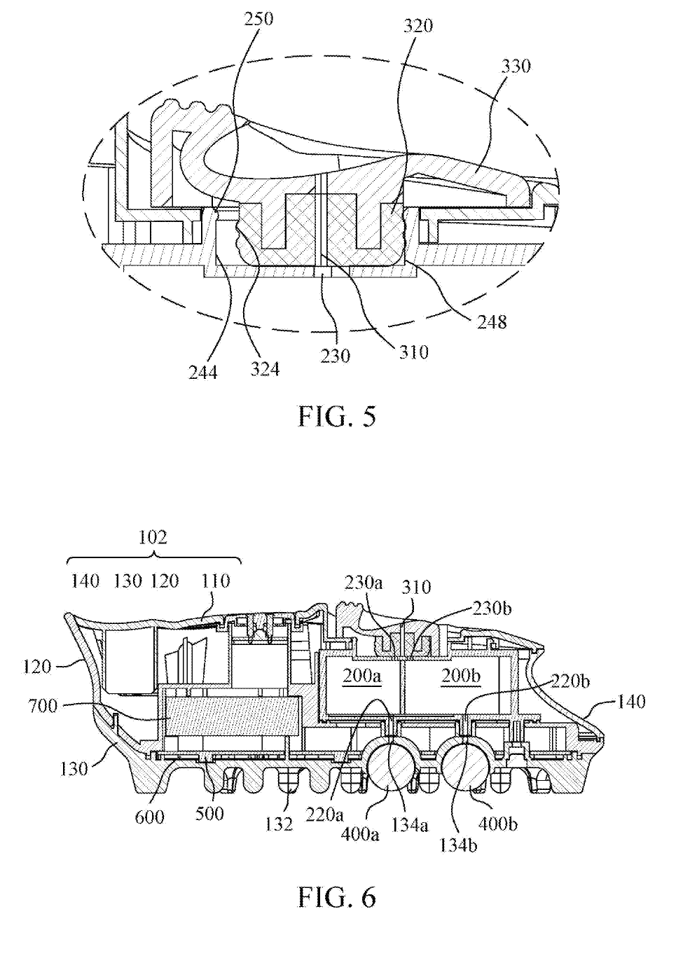

Referring to FIGS. 1 to 5, the physiotherapy comb device comprises a main body 100 and a sealing cover 300. The main body 100 includes a liquid chamber 210 for holding the care solution or medicament liquid. This liquid chamber 210 is in communication with a liquid outlet 220 and a first vent hole 230. The sealing cover 300 is configured to be installed on the main body 100 in a sliding manner along a first direction and can be switched between a first position and a second position. In the first position, the second vent hole 310 formed in the sealing cover 300 aligns with the first vent hole 230 in the main body 100, enabling air to enter the liquid chamber 210 through the two vent holes and allowing the care solution or medicament liquid to be smoothly discharged from the liquid outlet 220. When the sealing cover 300 is moved to the second position, the second vent hole 310 becomes staggered relative to the first vent hole 230, thereby blocking it and sealing the liquid chamber 210. This structure enables simple and reliable control over the dispensing of the care solution or medicament liquid, facilitating user operation.

The main body 100 is provided with a liquid chamber 210 that holds the care solution or medicament liquid. The liquid chamber 210 is connected to a liquid outlet 220 and a first vent hole 230, allowing the interior of the liquid chamber 210 to communicate with the outside air. When the liquid chamber 210 is filled with care solution or medicament liquid, the pressure inside and outside the chamber differs due to gravity pulling the liquid downward. When the first vent hole 230 is opened, air enters the liquid chamber, balancing the pressure difference and enabling a smooth and continuous outflow of the care solution or medicament liquid from the outlet. Thus, this design allows the care solution or medicament liquid to be dispensed through the liquid outlet 220 by gravity alone, ensuring a gentle and consistent flow rate without relying on valves or more complex dispensing structures.

When the care solution is used in combination with a physiotherapy comb device, the liquid outlet 220 is often set to be smaller so that the care solution flows out slowly from the liquid outlet 220, and the care solution can slowly act on the human body. However, when the liquid cavity only has the liquid outlet 220 and the liquid outlet 220 is smaller, the liquid chamber 210 is only affected by the atmospheric pressure at the liquid outlet 220. The atmospheric pressure can balance the gravity of the care solution, so that the care solution will not flow out or flow out unsmoothly, greatly reducing the user's experience. When the liquid chamber 210 adds the first vent hole 230 to connect with the atmospheric pressure, the liquid chamber 210 is affected by the atmospheric pressure at both liquid levels. The two air pressures offset each other, and due to the effect of gravity, the care solution can flow out from the liquid outlet 220.

The sealing is performed to maintain the airtightness of the liquid chamber 210 loaded with the care liquid. At this time, even if the liquid outlet 220 is open, the care liquid will basically not flow out due to the atmospheric pressure, or a plug can be added to block the liquid outlet 220 to completely prevent the liquid from flowing out of the liquid outlet 220. When the care liquid needs to be used, the sealing cover 300 can be pushed in the first direction to make the first vent hole 230 on the liquid chamber 210 communicate with the outside through the second vent hole 310 on the sealing cover 300. The outside gas enters the liquid chamber 210 from the first vent hole 230, which can destroy the balance at the liquid outlet 220, so that the care liquid can flow out smoothly from the liquid outlet 220 under the action of gravity, thereby improving the user experience.

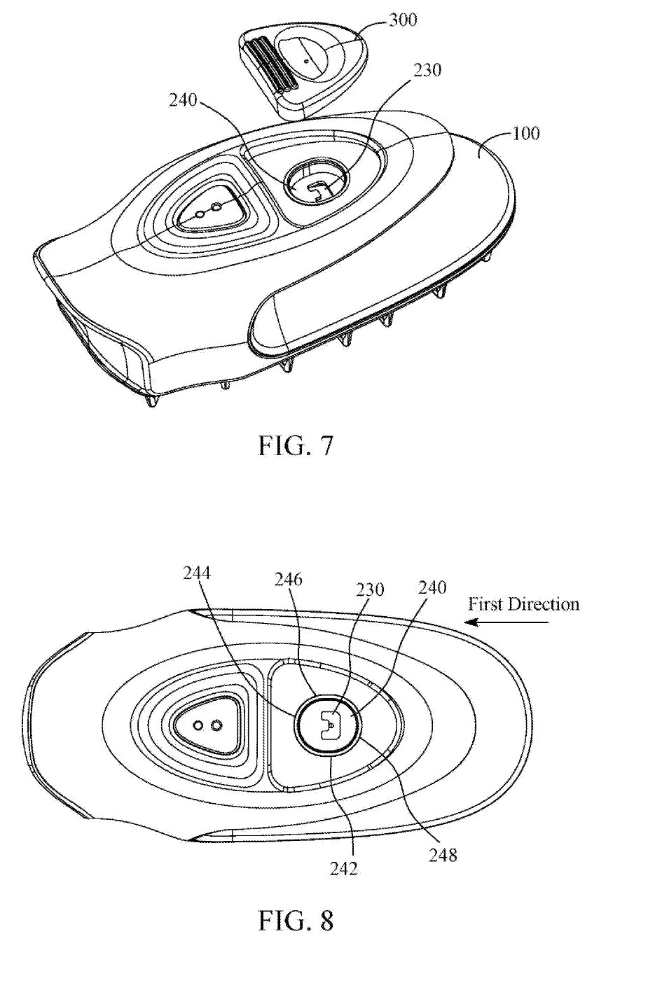

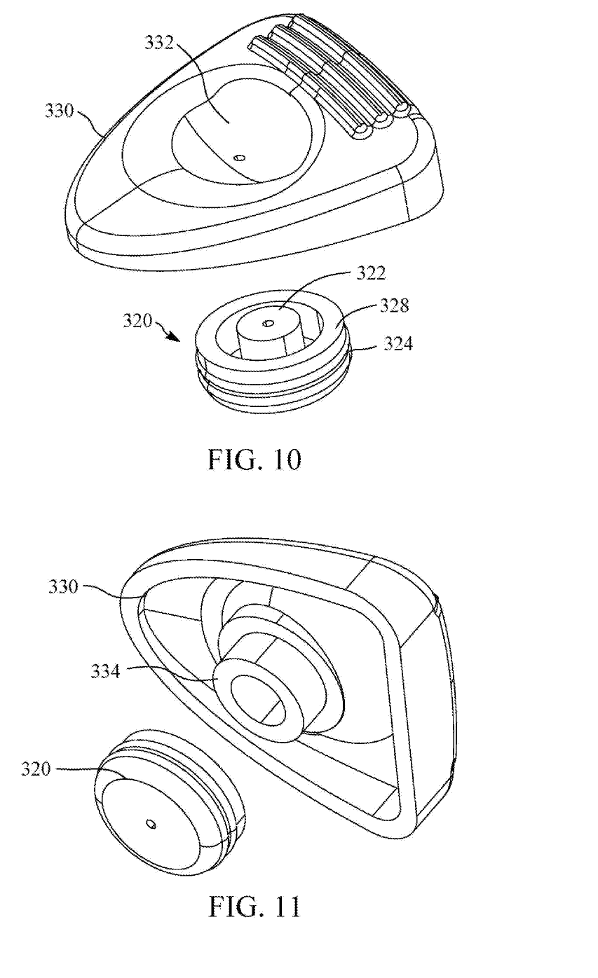

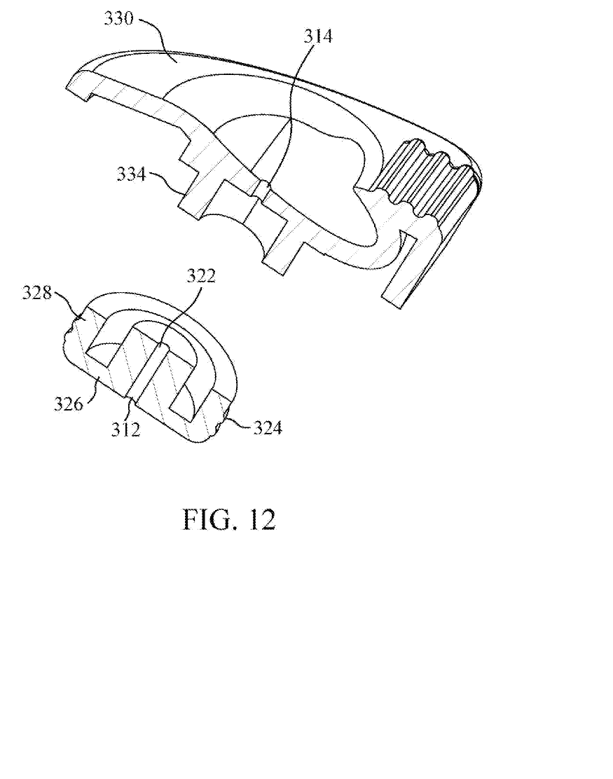

Referring to FIGS. 7 to 8 and FIGS. 10 to 12, the outer surface of the main body 100 is provided with a recessed active groove 240. A sealing part 320 is embedded in this active groove 240 and can slide within it along the first direction. The active groove 240 comprises four surrounding side walls: a first groove side wall 246, a second groove side wall 242, a third groove side wall 248, and a fourth groove side wall 244. The first groove side wall 246 and the second groove side wall 242 face each other and together clamp the sealing part 320 in the second direction, effectively preventing the sealing part 320 from becoming detached from the active groove 240 during sliding. The third groove side wall 248 and the fourth groove side wall 244 serve as end stops in the first direction, restricting the sliding range of the sealing part 320. In the first position, the sealing part 320 abuts against the third groove side wall 248. In the second position, it abuts the fourth groove side wall 244. This arrangement allows for precise switching between vented and sealed states based on the alignment of the vent holes.

The active groove 240 is further provided with a first latching protrusion 250 that protrudes toward the second direction, and the sealing part 320 is provided with a second latching protrusion 324. The first latching protrusion 250 and the second latching protrusion 324 face each other and jointly restrict movement of the sealing part 320 in the second direction, ensuring it remains confined within the active groove 240. This clamping design provides a certain degree of resistance during sliding, preventing the sealing part 320 from sliding too freely or being easily displaced. In actual use, the user can perceive a slight click or resistance, indicating successful engagement, and thereby simplifying the operation. This eliminates the need for a separate positioning structure.

The sealing part 320 is preferably circular, and the active groove 240 is preferably oval-shaped. The major axis of the oval aligns with the first direction, and the minor axis is shorter than the diameter of the sealing part 320. In this configuration, the sealing part 320 can slide back and forth within the groove’s lengthwise direction while remaining securely clamped in the widthwise direction. This arrangement ensures both freedom of movement and structural stability.

The sealing cover 300 includes a trigger part 330 and the sealing part 320. The trigger part 330 is fixedly connected to the sealing part 320 and controls its sliding movement. The trigger part 330 is provided with a ring-shaped protrusion 334 protruding in the second direction. The sealing part 320 includes a bottom wall 326, a center protrusion 322, and an edge protrusion 328. The edge protrusion 328 surrounds the center protrusion 322 and defines a ring groove that receives the ring-shaped protrusion 334. This design facilitates the integration of the sealing part 320 with the trigger part 330 and ensures smooth coordinated movement during actuation.

The second vent hole 310 includes a first ventilation section 314 formed through the trigger part 330 and a second ventilation section 312 formed through the center protrusion 322 and the bottom wall 326 of the sealing part 320. When the trigger part 330 is assembled to the sealing part 320, the first and second ventilation sections align, forming a complete and continuous ventilation path, a second vent hole 310 through the sealing cover 300.

The trigger part 330 is preferably made of a hard material such as ABS or PC, while the sealing part 320 is made of a soft material such as silica gel or rubber. Alternatively, the trigger part 330 and the sealing part 320 can be formed as an integral unit using dual-material injection molding technology, providing both rigidity and sealing flexibility in a single component.

In some embodiments, the first vent hole 230 can also be used as a filling port. In such cases, the sealing cover 300 is detachably mounted to the main body 100. By removing the sealing cover 300, the first vent hole 230 becomes exposed, allowing the user to pour in the care solution or medicament liquid directly. This configuration simplifies the overall structure by eliminating the need for a separate filling port.

The sealing cover 300 may be attached to the main body 100 by means of a snap fit or direct plug-in. A handle portion 332 may be formed on the trigger part 330. This handle portion may include grooves or protrusions that enhance grip and facilitate manual operation.

The liquid chamber 210 may be formed integrally with the main body 100 to simplify structural design. Alternatively, to improve ease of maintenance or enable more precise manufacturing, the liquid chamber 210 may be part of a container 200 that is housed within the main body 100.

Referring to FIG. 6, the physiotherapy comb device includes a first liquid container 200a and a second liquid container 200b, housed within the main body 100. Each liquid container is configured to store a distinct care solution, such as a hair growth serum, essential oil, scalp therapy liquid, or medicament. The first liquid container 200a is fluidly connected to a first liquid outlet 220a via a first vent hole 230a, and the second liquid container 200b is fluidly connected to a second liquid outlet 220b through a third vent hole 230b. The removable multi-container or multi-chamber arrangement allows the user to selectively dispense different solutions using a single device, thereby expanding the therapeutic versatility and convenience during personal care routines.

Alternatively, the sealing cover 300 may incorporate a common second vent hole 310, which is designed to align selectively with the first vent hole 230a and third vent hole 230b of the first liquid container 200a and the second liquid container 200b, depending on the sealing cover’s position. The sealing cover 300 is movable between at least three distinct positions: a first position, a second position, and a third position. In the first position, the second vent hole 310 aligns with the first vent hole 230a of the first liquid container 200a, permitting external air to enter and enabling gravity-assisted dispensing of the first care solution or medicament liquid from a first liquid outlet 220a. The first liquid outlet 220a is aligned with a first opening 134a, which further exposes the care solution or medicament liquid on a first rolling element 400a.

In the third position, the second vent hole 310 instead aligns with the third vent hole 230b of the second liquid container 200b, allowing dispensing of the second care solution from the second liquid outlet 220b. The second liquid outlet 220b is aligned with a third opening 134b, which then exposes the care solution or medicament liquid on a second rolling element 400b, allowing the care solution or medicament liquid to treat the user’s scalp.

In the second position, the second vent hole 310 is purposefully offset from both the first vent hole 230a and the third vent hole 230b of the first liquid container 200a and the second liquid container 200b. The offset condition ensures that neither liquid container is able to receive the atmospheric pressure over the liquid stored in the container, thus maintaining both liquid containers in a sealed state. The sealing cover 300 in this configuration effectively blocks airflow to both the first liquid container 200a and the second liquid container 200b, preventing unintentional leakage or evaporation when the device is not in active use. This second position acts as a secure “off” state, maintaining the longevity and stability of the stored care solutions or medicament liquid.

Additionally, the sealing cover 300 is configured to slide into a fourth position, wherein the second vent hole 310 simultaneously aligns with both the first vent hole 230a and third vent hole 230b. The dual alignment of the vent holes enables concurrent airflow into the first liquid container 200a and the second liquid container 200b. As a result, the care solution or medicament liquid from both containers is dispensed simultaneously and can be applied as a mixture of one or more care solutions or medicament liquids. The configuration supports the combined therapeutic application of multiple formulations during a single treatment session.

In an embodiment, the position of the sealing cover 300 can be adjusted relative to the first vent hole 230a and the third vent hole 230b. The sealing cover can also be positioned in a way that the second vent hole 310 aligns with a smaller portion of the first vent hole 230a and a larger portion of the third vent hole 230b, which results in more air pressure in the second liquid container 200b and lesser air pressure in first liquid container 200a, hence dispensing more amount of liquid from second liquid container 200b and minimal or lesser amount of liquid from first liquid container 200a. Through this feature, user can adjust both the position of the sealing cover 300 and the dispensing quantity of medicament liquid from each container according to their requirements.

Optionally, additional vent holes and corresponding positions may be integrated into the design to accommodate more than two liquid containers or chambers. The sealing cover 300 may include an extended sealing path with multiple air-passing apertures or vent holes, or an indexed sliding mechanism that allows users to cycle through multiple alignment configurations. Each configuration corresponds to a specific container, allowing precise selection of the desired solution for dispensing. This modular and expandable design not only enhances functionality but also provides a customizable and user-friendly interface for multi-step therapeutic treatments using a single physiotherapy comb device.

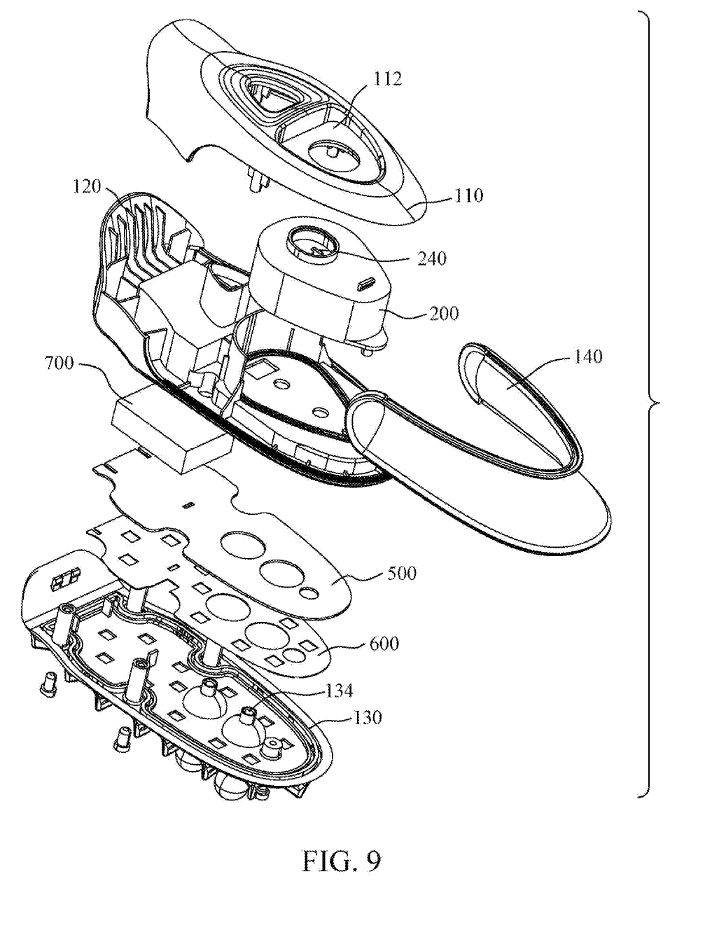

As shown in FIG. 4 and FIG. 9, the container 200 of the physiotherapy comb device comprises a liquid chamber 210, a liquid outlet 220, and a first vent hole 230. The container 200 is embedded within a housing 102. The housing 102 is provided with a first opening 134 that exposes the rolling element 400, and a second opening 112 that exposes the trigger part 330. The rolling element 400 is rotatably connected to the housing 102 and can rotate during application. When the user moves the device over the treatment area, the rolling element 400 rolls, providing a massage effect while dispensing the care solution or medicament liquid. This dual function enhances the therapeutic impact and improves the overall user experience.

The housing 102 is further provided with multiple protrusions, such as comb teeth 132 that serve as massage ridges. When the physiotherapy comb device is applied with a back-and-forth motion, these ridges rub against the treatment area, enhancing the massage effect. Meanwhile, the care solution dispensed from the liquid outlet 220 reduces surface friction and improves comfort. The comb teeth 132 may also be comb-shaped, enabling the physiotherapy comb device to function as a comb for human or pet use, facilitating scalp care and solution application while providing a soothing massage.

The physiotherapy comb device may also include at least one stimulation element that enhances the treatment experience. The stimulation element 500 may include, but not be limited to, a light therapy lamp, which emits red, blue, or purple light for phototherapy; a hot or cold compress element for temperature-based therapy; a microcurrent element for delivering electrical stimulation; or a vibration element to generate mechanical vibration. These stimulation elements may be activated individually or in combination, depending on the intended therapeutic effect.

In an embodiment, the stimulation element may include a pair of microcurrent electrodes disposed adjacent to or integrated with the comb teeth 132, which activate upon contact with the skin to deliver low-intensity electrical stimulation. In another configuration, the stimulation element may include a vibration motor, which transmits oscillatory motion through the main body 100 to facilitate scalp massage during use. In yet another embodiment, the stimulation element 500 may include a piezoelectric element to provide an electro-mechanical stimulus to the soft tissues of the user’s scalp. In another embodiment, the stimulation element 500 may include an ultrasonic wave therapy element to provide ultrasonic wave therapy to the user’s scalp to stimulate hair growth and improve scalp health. In yet another embodiment, the stimulation element 500 may further include a thermal element, such as a heating or cooling element, configured to facilitate hot compression or cold compression therapy for the user’s scalp.

The heating and/or cooling elements are configured to alter the temperature of the liquid stored within the container before application. The heating element may utilize a resistive heating element embedded near the liquid cavity, capable of raising the liquid temperature to a predetermined therapeutic range. Similarly, a thermoelectric cooling unit or Peltier chip may be incorporated to cool the liquid. These temperature variations can be selected based on user preference or treatment protocol, such as warm liquid to improve blood circulation or cool liquid to reduce inflammation or soothe irritation. Additionally, the heating and cooling element can heat/cool the surface having the comb teeth.

In some embodiments, the device is designed to pre-treat the liquid prior to dispensing through the liquid outlet. This pre-treatment may involve heating or cooling, or may include additional operations such as light irradiation using UV or near-infrared wavelengths to sterilize or activate specific therapeutic agents in the liquid. The liquid can be circulated through a light-exposure channel or chamber within the housing before it exits via the outlet. This ensures that the user receives a consistent and enhanced therapeutic dose during application.

To further augment the therapeutic benefits, the device may be equipped with a stimulation element 500 that may include multiple light-emitting diodes (LEDs) or a laser or a light source strategically positioned in the light therapy lamp. The LEDs can be aligned with the comb teeth 132 or placed adjacent to the comb teeth 132 or the liquid outlet region. These LEDs may emit light in various wavelengths, such as red light for anti-inflammatory effects, blue light for acne treatment, or near-infrared light for deep tissue stimulation. The LEDs can be configured to operate continuously, in pulsed/continuous mode, or in combination with other functional elements like heating or vibration, enabling comprehensive and customizable phototherapy treatments.

The housing 102 includes an upper shell 110, a middle shell 120, a lower shell 130, and a side shell 140. These components are vertically connected to accommodate the container 200 and the electronic modules. The stimulation element 500 or the light therapy lamp is mounted between the middle shell 120 and the lower shell 130. The lower shell 130 is preferably made of light-transmitting material, allowing the light emitted from the lamp to reach the skin surface. A concealing piece 600 is provided between the middle shell 120 and the lower shell 130 to prevent the internal structure of the housing 102 from being exposed to the outside through the lower shell 130, which is made of light-transmitting material. The concealing piece 600 is provided with light-transmitting holes that correspond to the lamp beads on the stimulation element 500 or the light therapy lamp, ensuring both functional and aesthetic integration of the light therapy element.

A battery 700 is housed between the middle shell 120 and the lower shell 130. The battery 700 supplies power to the stimulation element 500 or the light therapy lamp. The battery 700 and the container 200 are arranged side by side along the first direction to optimize internal space and enhance the overall compactness of the device.

The stimulation element 500 may be implemented as a single functional unit or as a combination of multiple elements within the container 200, allowing the device to deliver one or more therapeutic effects simultaneously or sequentially. Such combinations may include vibration, heating, cooling, phototherapy, ultrasonic stimulation, piezoelectric, and microcurrent therapy.

The main body 100 of the device may also include a vibration element integrated adjacent to the comb teeth 132 or the rolling element 400. The vibration element is capable of delivering gentle pulsations or continuous micro-vibrations during operation. The vibration feature enhances the penetration of the liquid into the scalp or skin, stimulates local blood flow, and provides a massaging effect that augments the therapy. The vibration settings may be user-adjustable, offering different frequencies or intensities based on the desired effect.

The physiotherapy comb also includes an intelligent control module housed within the main body 100, which governs the operation of all functional units, including the heating/cooling element, vibration unit, LED light array, laser, and liquid dispensing mechanism. The control module may be equipped with wireless communication capabilities, such as Bluetooth or Wi-Fi, allowing it to connect seamlessly with a dedicated application or user interface. Through this interface, users can monitor treatment progress, configure personalized therapy routines, adjust intensity levels, track usage history, and receive alerts or feedback. The integration of a user interface transforms the device into a smart therapeutic tool suitable for modern wellness management.

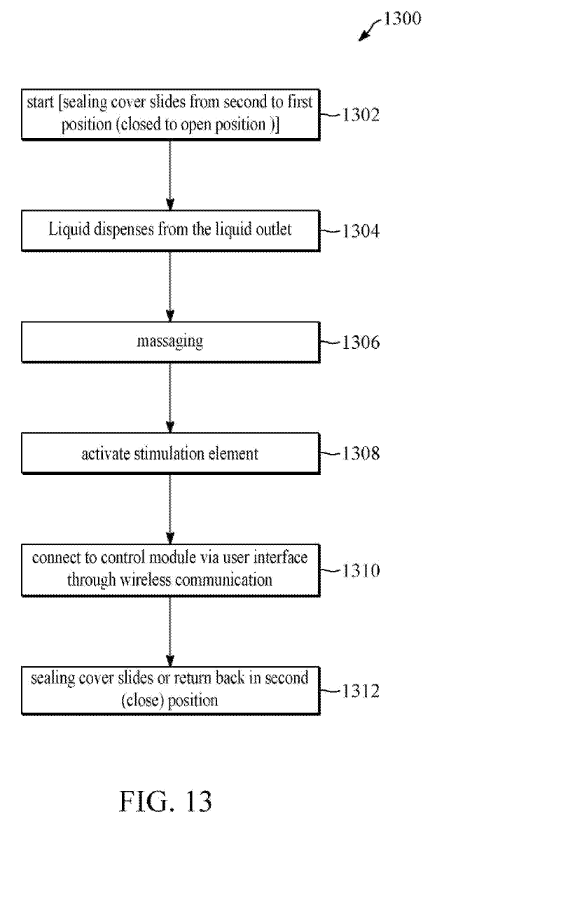

Referring to FIG. 13, a flowchart 1300 explaining the methodology to provide simultaneous topical application of a care solution and therapeutic stimulation to a user’s skin or scalp using a physiotherapy comb. The method comprises a process that begins with the step of providing the physiotherapy comb device, which includes a main body comprising at least one liquid chamber, a sealing cover configured with a second vent hole, a rotatable rolling element positioned near a liquid outlet, and one or more embedded stimulation elements such as light-emitting diodes (LEDs) or laser, vibration element, heating or cooling elements, Magnetic element, ultrasonic wave therapy element and microcurrent electrodes.

In an embodiment, the method further proceeds with step 1302, the user sliding the sealing cover from a second (closed) position to a first (open) position along a predefined linear direction. In this first position, the second vent hole formed in the sealing cover aligns with a first vent hole located on the liquid chamber. The alignment of the second and the first vent hole at step 1304 allows ambient air to enter the liquid chamber, thereby neutralizing internal vacuum pressure and enabling gravity-assisted flow of the care solution through the liquid outlet. The sealing cover is preferably guided along an active groove formed on the outer surface of the main body, and the sliding motion may be accompanied by tactile feedback to indicate correct positioning.

Once the device is in the first (open) position and dispensing is enabled, the user places the device in contact with the treatment area, typically the scalp or skin, and initiates application by moving the comb across the surface at step 1306. As the device is moved, the rolling element mounted adjacent to the liquid outlet rotates in response to surface contact and motion. The rolling element may be spherical or cylindrical and serves to evenly spread the dispensed care solution across the treatment area, while also delivering a mild massaging effect that enhances absorption and user comfort.

Simultaneously or subsequently, at step 1308, the stimulation elements embedded within the device can be activated. The stimulation elements may include phototherapy sources (such as red or infrared LEDs, laser), microcurrent electrodes, ultrasonic wave therapy elements for neuromuscular stimulation, vibratory motors for massage therapy, and thermal elements for delivering localised heating or cooling. The stimulation elements can be provided individually or in various combinations, depending on the therapeutic objectives. The stimulation enhances treatment outcomes by improving blood circulation, increasing cellular responsiveness, and facilitating deeper penetration of the applied care solution.

Furthermore, at step 1310, the method includes the optional use of an intelligent control module integrated into the comb device. The control module may support wireless communication protocols such as Bluetooth or Wi-Fi, allowing it to interface with an external user device, such as a mobile phone or tablet, via a dedicated application. Through the user interface, the user may customize treatment parameters including intensity, duration, light wavelength selection, stimulation pattern, and thermal settings. The user application may also provide real-time feedback, treatment logs, and usage statistics, thereby transforming the device into a smart, user-adaptive therapeutic tool.

The method concludes at step 1312 with the sealing cover being returned to the second (closed) position once treatment is completed. In this position, the second vent hole is displaced relative to the first vent hole, thereby sealing the liquid chamber and preventing unintended outflow or contamination of the care solution, ensuring long-term storage stability and hygiene. The rolling element and comb teeth may be cleaned or sanitised between uses, particularly in embodiments where the comb teeth are detachable.

The physiotherapy comb device, as described in the foregoing embodiments, provides a practical, user-friendly, and multifunctional tool suitable for use in a wide range of industries. In the personal care and beauty industry, the device supports applications such as scalp rejuvenation, hair serum delivery, phototherapy-based acne or inflammation treatments, and cosmetic dermatology procedures. In the medical and therapeutic sectors, the device may be employed for targeted skin treatments, transdermal drug delivery, and physiotherapeutic interventions involving vibration, microcurrent, or thermal modulation. In the pet care and veterinary domain, it may be adapted for fur grooming, antiseptic or moisturizing treatment delivery, and sensory stimulation. Its ergonomic design, gravity-fed dispensing mechanism, and integrated stimulation modules ensure that treatments can be applied evenly, efficiently, and with minimal user training or error.

The dual-container embodiment further broadens the industrial utility of the device, enabling sequential or alternating application of two separate liquid solutions, such as pre- and post-treatment agents, or day and night formulas, within a single compact unit. This makes the device particularly attractive to professional practitioners and home users alike who seek versatile yet intuitive therapeutic tools.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A comb device comprising:
a main body including a housing and a liquid chamber configured to store a care solution, the liquid chamber being in communication with a liquid outlet and a first vent hole;
a sealing cover comprising a sealing part and a trigger part, the sealing cover being slidably mounted on the main body between a first position and a second position;
wherein in the first position, a second vent hole formed in the sealing cover aligns with the first vent hole to allow airflow into the liquid chamber for dispensing the care solution through the liquid outlet;
wherein in the second position, the second vent hole is offset from the first vent hole, thereby sealing the liquid chamber;
a rolling element rotatably mounted on the housing and positioned adjacent the liquid outlet, configured to dispense the care solution; and
at least one stimulation element embedded in the housing to provide a therapy to a user.

2. The comb device of claim 1, wherein the main body includes an active groove formed on an outer surface, and the sealing part of the sealing cover is configured to be received and slidable within the active groove along the first direction.

3. The comb device of claim 2, wherein the active groove comprises opposing first and second groove side walls that elastically clamp the sealing part to restrict movement in a direction perpendicular to the first direction.

4. The comb device of claim 1, wherein the first vent hole has a diameter larger than the second vent hole, and wherein the first vent hole also functions as a refill port when the sealing cover is removed.

5. The comb device of claim 1, wherein the sealing cover comprises a trigger part having a rigid structure and a sealing part made of an elastomeric material, and is joined via dual-material injection molding.

6. The comb device of claim 5, wherein the trigger part includes a ring-shaped protrusion embedded in a ring groove formed between a centre protrusion and an edge protrusion of the sealing part.

7. The comb device of claim 1, wherein the rolling element is spherical or cylindrical and seated in a recessed socket of the main body, configured to rotate and evenly distribute the care solution across a user’s scalp or skin.

8. A comb device of claim 1, wherein the at least one stimulation element includes at least one light-emitting diode (LED) or laser, micro-current electrode, heating element, cooling element, vibration element, piezoelectric element, or ultrasonic wave therapy element.

9. The comb device of claim 1, further comprising a plurality of comb teeth extending from the housing, the comb teeth including grooves or microchannels for guiding dispensed liquid toward user’s skin or scalp.

10. The comb device of claim 9, wherein the plurality of comb teeth are formed of a light-transmitting polymer and are configured to deliver light from the stimulation element directly to a treatment site.

11. The comb device of claim 9, wherein the plurality of comb teeth are detachably connected to the main body via a magnetic or snap-fit coupling to facilitate cleaning or replacement.

12. A comb device comprising:
a main body including a housing;
a first liquid container configured to store a first care solution, the first liquid container being in communication with a first liquid outlet and a first vent hole;
a second liquid container configured to store a second care solution, the second liquid container being in communication with a second liquid outlet and a second vent hole;
a sealing cover comprising a sealing part and a trigger part, the sealing cover being slidably mounted on the housing and movable between a first position, a second position, and a third position;
wherein in the first position, a third vent hole formed in the sealing cover aligns with the first vent hole of the first liquid container, allowing airflow into the first liquid container and enabling dispensing of the first care solution through the first liquid outlet;
wherein in the second position, the third vent hole is offset from both the first vent hole and the second vent hole of the respective liquid containers, thereby sealing both liquid containers;
wherein in the third position, the third vent hole aligns with the second vent hole of the second liquid container, allowing airflow into the second liquid container and enabling dispensing of the second care solution through the second liquid outlet; and
a rolling element rotatably mounted on the housing and positioned adjacent the first liquid outlet or the second liquid outlet, the rolling element being configured to dispense at least one of the care solutions during application.

13. The comb device of claim 12, wherein the first liquid container and the second liquid container are removably mounted within the housing and are configured to be individually refillable or replaceable.

14. The comb device of claim 12, wherein the sealing cover is slidable within an active groove formed on the housing, and the active groove comprises a latching or detent mechanism to provide tactile feedback during position changes.

15. The comb device of claim 12, wherein each of the first liquid outlet and the second liquid outlet is associated with corresponding rolling elements that enable selective dispensing of the respective care solutions during movement.

16. The comb device of claim 12, further comprising at least one stimulation element to provide therapy to a user.

17. A method of performing a therapeutic treatment using a comb device, the method comprising:
providing a comb device comprising: a main body having a housing and at least one liquid chamber in fluid communication with a liquid outlet and a first vent hole, a sealing cover slidably mounted on the main body, the sealing cover having a second vent hole;
a rolling element rotatably mounted adjacent to the liquid outlet;
moving the sealing cover to a first position such that the second vent hole aligns with the first vent hole, allowing air to enter the liquid chamber and enabling gravity-assisted dispensing of a care solution through the liquid outlet; and
rotating the rolling element against a treatment surface while dispensing the care solution, the rolling element spreading the care solution onto user’s skin or scalp.

18. The method of claim 17, wherein the rolling element is a spherical or cylindrical massaging ball that rotates during movement of the device across user's skin or scalp to simultaneously spread the care solution and apply mechanical stimulation.

19. The method of claim 17, further comprising activating a stimulation element embedded in the main body, wherein the stimulation element delivers at least one therapy.

20. The method of claim 19, further comprising guiding the care solution trough one or more grooves formed along a plurality of comb teeth extending from the main body, wherein the plurality of comb teeth are formed of a light-transmitting material and the stimulation element emits light that travels through the comb teeth to deliver localized phototherapy.
